(19) United States
(12) Patent Application Publication
(10) Pub. No.: US 2025/0352398 A1
(43) Pub. Date: Nov. 20, 2025

(54) CHEST SEAL

(71) Applicant: Pneumeric, Inc., Rochester, MN (US)

(72) Inventors: Johnathon Aho, Rochester, MN (US); Nathan Wells, Wichita, KS (US)

(21) Appl. No.: 19/078,984

(22) Filed: Mar. 13, 2025

Related U.S. Application Data
(60) Provisional application No. 63/648,444, filed on May 16, 2024.

(51) Int. Cl. A61F 13/14; A61F 13/00; A61F 13/02

(52) U.S. Cl. CPC A61F 13/143; A61F 13/00055; A61F 13/0233; A61F 2013/00114

(57) ABSTRACT

A chest seal includes a seal body, a hub port at the seal body, at least one main channel at the seal body, and a first secondary channel at the seal body. The hub port extends from a first seal body side toward a second seal body side. The at least one main channel is in fluid communication with the hub port, and the at least one main channel includes a main channel inlet at the hub port and a main channel outlet at a perimeter of the seal body. The first secondary channel includes a first secondary channel inlet at the at least one main channel and a first secondary channel outlet at the perimeter of the seal body. The first secondary channel outlet is spaced apart about the perimeter of the seal body from the main channel outlet.

### RELATED APPLICATION

This disclosure claims priority to U.S. provisional patent application No. 63/648,444, filed on May 16, 2024, the entire contents of which are hereby incorporated by reference.

### TECHNICAL FIELD

This disclosure is generally directed to device, system, and method embodiments relating to chest seals. Chest seal embodiments disclosed herein can be used, for instance, to help alleviate pneumothorax, such as to treat traumatic pneumothorax in a prehospital setting.

### BACKGROUND

In 2021 nearly 225,000 deaths occurred as a result of preventable trauma in the United States. According to some reports, thoracic injuries account for 25-32% of all traumatic injuries. Thoracic injuries are also a major threat in combat scenarios. During the recent US military operations in the Middle East, thoracic injuries accounted for 4.9%-9.9% of all combat-related injuries, around 10% of which proved to be fatal. A study of a large combat casualty database from the Vietnam War estimated 3%-4% of all combat casualties were a result of tension pneumothorax, many of which may have been treatable. It was also found that a significant amount of blood filled the chest cavity in about half of the identified thoracic injuries, highlighting the importance of draining both air and blood for effective treatment of an open chest wound.

The treatment of a traumatic pneumothorax in a prehospital setting is of high urgency. Once the chest wall is punctured and the lung collapses, there is a threat of developing a tension pneumothorax as air and fluid fill and become trapped in the chest wall. The deviation of the lung from the chest wall only needs to reach two-thirds the size of the trachea before air will preferentially enter the lung through the chest wound instead of the trachea. Treatment options have included completely sealing the chest wound with an occlusive dressing, securing a dressing only on three sides, or using a more complex seal to create a one-way valve vented seal which can allow for air and fluid drainage from the chest cavity while keeping air from entering through the chest wound. While evidence suggests occlusive dressings initially restore respiratory mechanics, it can often eventually lead to development of tension pneumothorax and respiratory failure if air continues to accumulate in the chest cavity. While currently available one-way valve vented seals have been shown to be capable of acting as a one-way vent to evacuate air from the chest cavity and restore respiratory function, in real world traumatic pneumothorax applications, as blood enters these currently available one-way valve vented seals, these currently available one-way valve vented seals are susceptible to air flow blockage as a result of blood clots formed within the seal.

### SUMMARY

This disclosure is generally directed to device, system, and method embodiments relating to chest seals. One exemplary application of chest seal embodiments disclosed herein can include the treatment of pneumothorax, such the treatment of traumatic pneumothorax in a prehospital setting.

Chest seal embodiments disclosed herein can include one or more main channels and one or more secondary channels at the seal body to help improve fluid (e.g., blood) drainage through the chest seal which, in turn, can help to improve the chest seal's ability to vent fluid (e.g., air) effectively and thereby treat pneumothorax. The one or more main channels and one or more secondary channels can be configured at the seal body to help separate different fluids—for instance to help separate blood and air—expired from an open chest wound. For instance, the one or more main channels and one or more secondary channels at the seal body can help to ensure that at least one or more main channels and/or one or more secondary channels do not fill with one type of fluid (e.g., blood in applications of the chest seal embodiment to hemopneumothorax or pleural effusion) and, thereby, help to reduce the chance of complete drainage blockage of another type of fluid (e.g., air, such as CO₂) at the chest seal due to clotting of the first fluid type (e.g., blood) within the chest seal which would prohibit the chest seal from effectively venting out the other fluid type (e.g., air). Notably, certain chest seal embodiments disclosed herein can be configured to help separate different fluids (e.g., blood and air) within the chest seal body without using any mechanically actuated component (e.g., without using a moving component, such as without using a mechanically actuated valve), which, in turn, can help to reduce cost and case manufacturing.

One chest seal embodiment disclosed herein includes a seal body, a hub port at the seal body, at least one main channel at the seal body, and a first secondary channel at the seal body. The hub port extends from a first seal body side toward a second seal body side. The at least one main channel is in fluid communication with the hub port, and the at least one main channel includes a main channel inlet at the hub port and a main channel outlet at a perimeter of the seal body. The first secondary channel includes a first secondary channel inlet at the at least one main channel and a first secondary channel outlet at the perimeter of the seal body. The first secondary channel outlet is spaced apart about the perimeter of the seal body from the main channel outlet.

In a further embodiment of this chest seal, the first secondary channel inlet is in direct fluid communication with the at least one main channel, and the first secondary channel inlet is in indirect fluid communication with the hub port via the direct fluid communication with the at least one main channel.

In a further embodiment of this chest seal, the chest seal further includes a second secondary channel defined at the seal body. The second secondary channel includes a second secondary channel inlet at the at least one main channel and a second secondary channel outlet at the perimeter of the seal body. The first secondary channel outlet can be spaced apart about the perimeter of the seal body from the second secondary channel outlet. In one such example, the first secondary channel extends along the seal body in a first radial direction from the first secondary channel inlet to the first secondary channel outlet at the perimeter of the seal body, and the second secondary channel extends along the seal body in a second radial direction from the second secondary channel inlet to the second secondary channel outlet at the perimeter of the seal body, where the second radial direction is different than the first radial direction.

In a still further embodiment of this chest seal, the first secondary channel inlet is in direct fluid communication with the at least one main channel, and the first secondary channel inlet is in indirect fluid communication with the hub port via the direct fluid communication with the at least one main channel. Similarly, the second secondary channel inlet is in direct fluid communication with the at least one main channel, and the second secondary channel inlet is in indirect fluid communication with the hub port via the direct fluid communication with the at least one main channel.

In one such example, the first secondary channel inlet intersects the at least one main channel at a first location along the main channel spaced apart from both the main channel inlet and the main channel outlet. Similarly in this example, the second secondary channel inlet intersects the at least one main channel at a second location along the main channel spaced apart from both the main channel inlet and the main channel outlet, where the second location is spaced radially about the seal body from the first location.

In another such example, the first secondary channel inlet intersects the at least one main channel at a first location along the main channel spaced apart from both the main channel inlet and the main channel outlet, and the second secondary channel inlet intersects the at least one main channel at the first location along the main channel spaced apart from both the main channel inlet and the main channel outlet. For instance, the first secondary channel can extend along the seal body in a first radial direction from the first secondary channel inlet at the first location to the first secondary channel outlet at the perimeter of the seal body. And the second secondary channel can extend along the seal body in a second radial direction from the second secondary channel inlet at the first location to the second secondary channel outlet at the perimeter of the seal body, where the second radial direction is different than the first radial direction.

In a still further embodiment of this chest seal, the first secondary channel inlet and the first secondary channel outlet define a same cross-sectional area, and the second secondary channel inlet and the second secondary channel outlet define a same cross-sectional area.

In a still further embodiment of this chest seal, the first secondary channel inlet and the first secondary channel outlet define different cross-sectional areas, and the second secondary channel inlet and the second secondary channel outlet define different cross-sectional areas.

In a still further embodiment of this chest seal, the first secondary channel inlet defines a first cross-sectional area and the second secondary channel inlet defines a second cross-sectional area that is different than the first cross-sectional area.

In a still further embodiment of this chest seal, the first secondary channel defines a first cross-sectional area along at least a portion of a first secondary channel length between the first secondary channel inlet and the first secondary channel outlet. And the at least one main channel defines a second cross-sectional area along at least a portion of a main channel length between the main channel inlet and the main channel outlet, where the first cross-sectional area is different than the second cross-sectional area.

In a still further embodiment of this chest seal, the at least one main channel is a first main channel, the main channel inlet is a first main channel inlet, and the main channel outlet is a first main channel outlet. This chest seal further includes a second main channel. The second main channel is defined at the seal body. The second main channel includes a second main channel inlet at the hub port and a second main channel outlet at the perimeter of the seal body. The second main channel outlet is spaced apart about the perimeter of the seal body from the first main channel outlet.

As one such example, the first main channel is in direct fluid communication with the hub port at the first main channel inlet, and the first main channel extends a first radial direction along the seal body from the first main channel inlet to the first main channel outlet. The second main channel is in direct fluid communication with the hub port at the second main channel inlet, and the second main channel extends a second radial direction along the seal body from the second main channel inlet to the second main channel outlet, where the first radial direction is different than the second radial direction. For instance, the first secondary channel inlet can be in direct fluid communication with the first main channel and can be in indirect fluid communication with the hub port via the direct fluid communication with the first main channel. Similarly, the second secondary channel inlet can be in direct fluid communication with the second main channel and can be in indirect fluid communication with the hub port via the direct fluid communication with the second main channel.

In a still further embodiment of this chest seal, the chest seal further includes at least one fluid sensor at the seal body. For example, the at least one fluid sensor can be positioned at at least one of the main channel outlet at the perimeter of the seal body and the first secondary channel outlet at the perimeter of the seal body. As one specific such example, the at least one fluid sensor includes a first fluid sensor and a second fluid sensor, with the first fluid sensor positioned at the main channel outlet at the perimeter of the seal body and the second fluid sensor positioned at the first secondary channel outlet at the perimeter of the seal body.

In a still further embodiment of this chest seal, the first seal body side includes an adhesive. The hub port is located at a center of the seal body, and the hub port extends longitudinally from the center of the seal body at the first seal body side toward the second seal body side.

The details of one or more examples are set forth in the accompanying drawings and the description below. Other features, objects, and advantages will be apparent from the description and drawings, and from the claims.

### BRIEF DESCRIPTION OF DRA WINGS

The following drawings are illustrative of particular examples of the present invention and therefore do not limit the scope of the invention. The drawings are intended for use in conjunction with the explanations in the following detailed description wherein like reference characters denote like elements. Examples of the present invention will hereinafter be described in conjunction with the appended drawings.

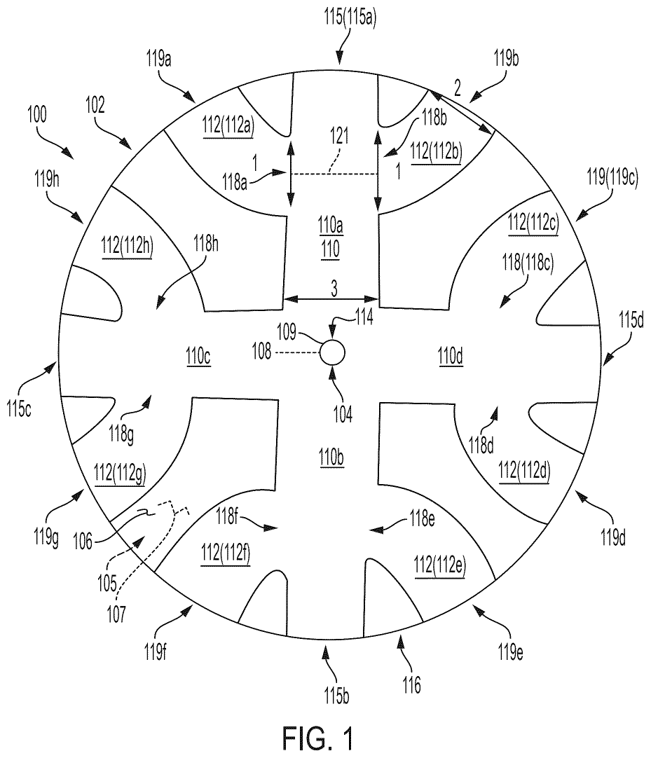

FIG. 1 is a top plan view, schematic representation of an embodiment of a chest seal.

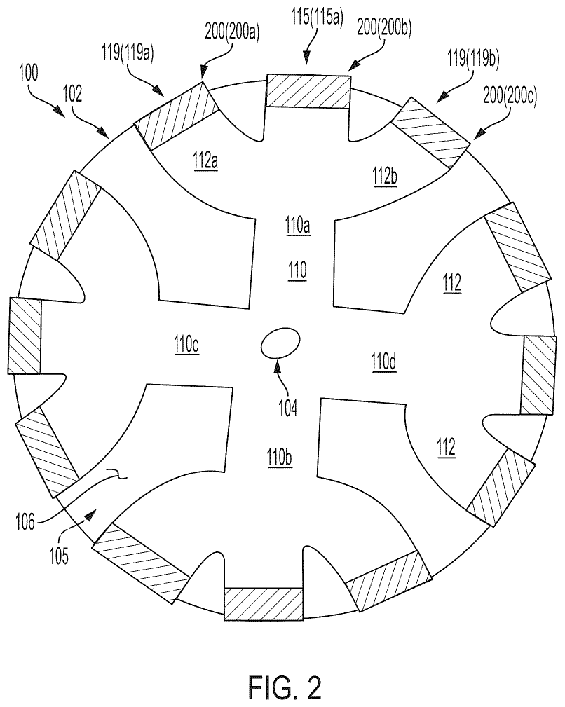

FIG. 2 is a top plan view, schematic representation of the chest seal embodiment of FIG. 1 with one or more fluid sensors included at the seal body.

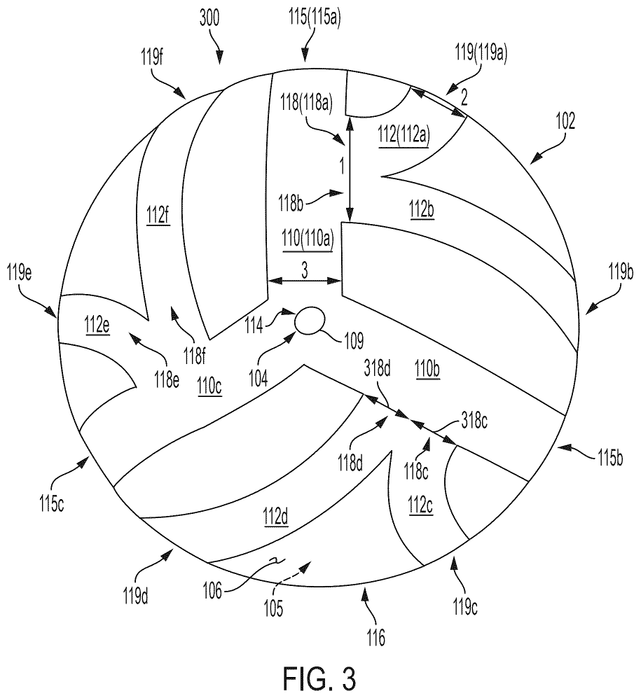

FIG. 3 is a top plan view, schematic representation of another embodiment of a chest seal.

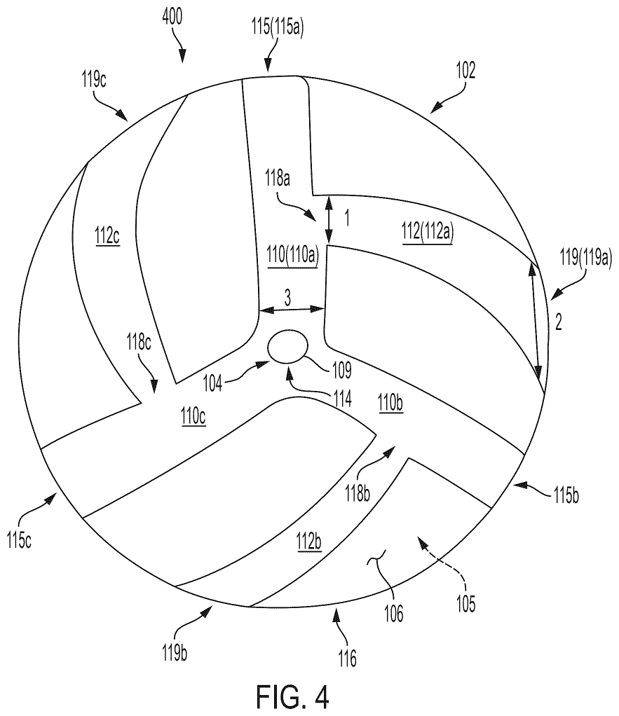

FIG. 4 is a top plan view, schematic representation of an additional embodiment of a chest seal.

### DETAILED DESCRIPTION

The following detailed description is exemplary in nature and is not intended to limit the scope, applicability, or configuration of the invention in any way. Rather, the following description provides some practical illustrations for implementing examples of the present invention. Those skilled in the art will recognize that many of the noted examples have a variety of suitable alternatives.

FIG. 1 illustrates a top plan view, schematic representation of an embodiment of a chest seal 100. The chest seal 100 incudes a seal body 102 and a hub port 104 at the seal body 102. In certain embodiments, the seal body 102 can be formed as an integrated single-layer body. The seal body 102 has a first seal body side 105 and a second seal body side 106 that is opposite the first seal body side 105. For example, the first seal body side 105 can be configured to interface (e.g., contact) with an anatomic surface of interest, such as a chest wound at a chest wall of a patient. To help adhere the seal body 102 to the anatomic surface of interest, the first seal body side 105 can include an adhesive 107 that is configured to adhere the seal body 102 to a skin surface of a patient.

The hub port 104 can extend from the first seal body side 105 toward the second seal body side 106. For example, the hub port 104 can extend longitudinally (e.g., in a direction into and/or out of the page at the plan view of FIG. 1) from the first seal body side 105 to the second seal body side 106. In one particular such example, the hub port 104 can extend longitudinally from a first seal body side inlet 108 at the first seal body side 105 to a second seal body side outlet 109 at the second seal body side 106. For instance, the illustrated embodiment shows the hub port 104 located at a radial center of the seal body 102 such that the hub port 104 extends longitudinally from the center of the first seal body side 105 at the first seal body side inlet 108 to the center of the second seal body side 106 at the second seal body side outlet 109. As such, the seal body 102 can be placed over a chest wound such that the first seal body side inlet 108 of the hub port 104 is placed over the chest wound and the hub port 104 is thus positioned to receive fluid (e.g., blood and/or carbon dioxide) from the chest wound via the first seal body side inlet 108, and the hub port 104 is configured to convey such received fluid through the hub port 104 to the second seal body side outlet 109.

The chest seal 100 also includes at least one main channel 110 defined at the seal body 102 and at least one secondary channel 112 defined at the seal body 102. The at least one main channel 110 and the at least one secondary channel 112 can be configured to receive fluid (e.g., blood and/or carbon dioxide) from the hub port 104 and configured to convey this received fluid radially along the seal body 102 to output such fluid from the seal body 102. The at least one main channel 110 can be in fluid communication with the hub port 104, and the at least one secondary channel 112 can be in fluid communication with the at least one main channel 110. The at least one main channel 110 can be configured to receive fluid directly from the hub port 104 (e.g., directly from the second seal body side outlet 109), while the at least one secondary channel 112 can be configured to receive fluid directly from the at least one main channel 110 and thus indirectly from the hub port 104. In other words, because the at least one secondary channel 112 may not be in direct fluid communication with the hub port 104, all fluid at the at least one secondary channel 112 may come directly from the at least one main channel 110 which serves as an intermediary to convey fluid from the hub port 104, through at least a portion of a length of the at least one main channel 110, and to the at least one secondary channel 112.

The inventors have discovered that this indirect fluid communication from the hub port 104 to the at least one secondary channel 112 can be useful to help improve fluid (e.g., blood) drainage through the chest seal 100 which, in turn, can help to improve the chest seal's ability to vent fluid (e.g., air, such as carbon dioxide) effectively and thereby treat pneumothorax. For instance, the inventors have discovered that this indirect fluid communication from the hub port 104 to the at least one secondary channel 112 can help separate different fluids—for instance to help separate blood and air (e.g., carbon dioxide)—expired from an open chest wound. The indirect fluid communication from the hub port 104 to the at least one secondary channel 112 via the direct fluid communication between the hub port 104 and the at least one main channel 110 can help to reduce instances where the at least one secondary channel 112 fills with one type of fluid (e.g., blood in applications of the chest seal embodiment to hemopneumothorax or pleural effusion) and, thereby, help to reduce the chance of complete drainage blockage of another type of fluid (e.g., air, such as CO₂) at the chest seal due to clotting of the first fluid type (e.g., blood) within the at least one secondary channel 112 at the chest seal body 102 which could prohibit the chest seal from effectively venting out the other fluid type (e.g., air).

The at least one main channel 110 can include a main channel inlet 114 and a main channel outlet 115. The main channel inlet 114 can be at the hub port 104, for instance at the second seal body side outlet 109, and the main channel outlet 115 can be at a perimeter 116 of the seal body 102. Accordingly, the at least one main channel 110 can be configured to receive fluid (e.g., blood and/or air) from a chest wound via the second seal body side outlet 109 of the hub port 104 and configured to convey such received fluid from the second seal body side outlet 109, through a length of the at least one main channel 110, to the main channel outlet 115 where such fluid can be output from the seal body 102.

The illustrated embodiment of the chest seal 100 includes four main channels 110—first main channel 110a extending from the second seal body side outlet 109 to a first main channel outlet 115a at a first perimeter location; second main channel 110b extending from the second seal body side outlet 109 to a second main channel outlet 115b at a second, different perimeter 116 location; third main channel 110c extending from the second seal body side outlet 109 to a third main channel outlet 115c at a third, different perimeter 116 location; and fourth main channel 110d extending from the second seal body side outlet 109 to a fourth main channel outlet 115d at a fourth, different perimeter 116 location. Though other embodiments within the scope of this disclosure can include other numbers of main channels 110, such as more than four main channels 110 or less than four main channels 110.

The at least one secondary channel 112 can include a secondary channel inlet 118 and a secondary channel outlet 119. The secondary channel inlet 118 can be at, and in direct fluid communication with, the main channel 110 such that the secondary channel 112 receives fluid directly from the main channel 110 (e.g., the secondary channel inlet 118 only receives fluid from the main channel 110 and not from the hub port 104). For instance, the secondary channel inlet 118 can be at the main channel 110 at a location along a length of the main channel 110 that is spaced apart from the second seal body side outlet 109 and spaced apart from the main channel outlet 115. The secondary channel outlet 119 can be at the perimeter 116 of the seal body 102. For instance, the secondary channel outlet 119 can be at the perimeter 116 and spaced apart from the main channel outlet(s) 115 also at the perimeter 116. Accordingly, the at least one secondary channel 112 can be configured to receive fluid (e.g., blood and/or air) directly from the at least one main channel 110 and configured to convey such received fluid from the main channel 110 at the secondary channel inlet 118 and through a length of the at least one secondary channel 110 from the secondary channel inlet 118 to the secondary channel outlet 119 where such fluid can be output from the seal body 102. Thus, the chest seal 100 can be configured such that each secondary channel inlet 118 is (i) in direct fluid communication with at least one main channel 110 and (ii) in indirect fluid communication with the hub port 104 via the direct fluid communication with the at least one main channel 110.

The illustrated embodiment of the chest seal 100 includes eight secondary channels 112—first secondary channel 112a extending from a first secondary channel inlet 118a at main channel 110a to a first secondary channel outlet 119a at a first perimeter 116 location; second secondary channel 112b extending from a second secondary channel inlet 118b at main channel 110a to a second secondary channel outlet 119b at a second perimeter 116 location; third secondary channel 112c extending from a third secondary channel inlet 118c at main channel 110d to a second secondary channel outlet 119c at a third perimeter 116 location; fourth secondary channel 112d extending from a fourth secondary channel inlet 118d at main channel 110d to a fourth secondary channel outlet 119d at a fourth perimeter 116 location; fifth secondary channel 112e extending from a fifth secondary channel inlet 118e at main channel 110b to a fifth secondary channel outlet 119e at a fifth perimeter 116 location; sixth secondary channel 112f extending from a sixth secondary channel inlet 118f at main channel 110b to a sixth secondary channel outlet 119f at a sixth perimeter 116 location; seventh secondary channel 112g extending from a seventh secondary channel inlet 118g at main channel 110c to a seventh secondary channel outlet 119g at a seventh perimeter 116 location; and eighth secondary channel 112h extending from a eighth secondary channel inlet 118h at main channel 110c to an eighth secondary channel outlet 119h at an eighth perimeter 116 location. Though other embodiments within the scope of this disclosure can include other numbers of secondary channels 112, such as more than eight secondary channels 112 or less than eight secondary channels 112.

For embodiments of the chest seal 100 that include two or more secondary channels 112, in some such examples the secondary channels 112 can extend in different directions along the seal body 102. For instance, the example of FIG. 1 shows pairs of branched configuration secondary channels 112 extending from a common main channel 110. Namely, secondary channel 112a and secondary channel 112b each branch out from common secondary channel 110a. Secondary channel 112a can extend a length along the seal body 102 in a first radial direction from secondary channel inlet 118a that is at main channel 110a to the secondary channel outlet 119a at the perimeter 116 of the seal body 102, and secondary channel 112b can extend a length along the seal body 102 in a second, different (e.g., opposite) radial direction from secondary channel inlet 118b that is at main channel 110a to the secondary channel outlet 119b at the perimeter 116 of the seal body 102. FIG. 1 shows that these pairs of branched secondary channels 112 can be spaced apart from one another about the seal body, for instance as shown for the illustrated embodiment which includes such pairs of branched secondary channels 112 spaced generally ninety degrees (e.g., as measured from a center of the pair of branched secondary channels 112) from one another about the perimeter 116.

Also for embodiments of the chest seal 100 that include two or more secondary channels 112, in some such examples the secondary channels 112 can be in direct fluid communication with the main channel(s) 110 at different locations along the main channel(s) 110 between the main channel inlet 114 and the main channel outlet 115. For example, first secondary channel 112a can have first secondary channel inlet 118a at a first location along main channel 110a while second secondary channel 112b can have second secondary channel inlet 118b at a second, different location along main channel 110a that is radially spaced along the seal body 102 from the first secondary channel inlet 118a. As an additional or alternate example, two or more secondary channel inlets 118 can intersect a same main channel 110 along a common axis. For instance, first secondary channel inlet 118a and second secondary channel inlet 118b can intersect main channel 110a along a common axis 121.

The size of the main channel(s) 110 and secondary channel(s) 112 can vary depending on the intended application of chest seal 100. For some embodiments, chest seal 100 can include main channel(s) 110 having a cross-sectional area greater than a cross-sectional area of secondary channel(s) 112. In other embodiments, chest seal 100 can include secondary channel(s) 112 having a cross-sectional area greater than a cross-sectional area of main channel(s) 110. For some additional or alternative embodiments, secondary channel inlet(s) 118 can have a cross-sectional area smaller or larger than a cross-sectional area of the main channel 110 where the secondary channel inlet 118 intersects the main channel 110 (e.g., secondary channel inlet(s) 118 can have a cross-sectional area smaller than a cross-sectional area of main channel inlet 114). For some additional or alternative embodiments, secondary channel inlet(s) 118 can have a cross-sectional area that is smaller or larger than a cross-sectional area of secondary channel outlet(s) 119 of the same secondary channel 112. As one example referring to the chest seal 100 embodiment shown at FIG. 1, secondary channel inlet 118b can have a cross-sectional area 1 that is the same as, greater than, or less than a cross-sectional area 2 of secondary channel outlet 119b. As further examples, the chest seal body 102 can have a diameter of 15.25 cm, the main channel(s) 110 can have a diameter 3 of approximately 2 cm, and the secondary channel(s) 112 can have a diameter 1 (e.g., at or near the secondary channel inlet 118) and/or 2 (e.g., at or near the secondary channel outlet 119) that is equal to, greater than, or less than the dimeter 3 of the main channel(s) 110. In some such examples, a total cross-sectional area of the main channel(s) 110 plus the secondary channel(s) 112 can be at least 10% of the total cross-sectional area of the seal body 102, at least 15% of the total cross-sectional area of the seal body 102, at least 20% of the total cross-sectional area of the seal body 102, or at least 25% of the total cross-sectional area of the seal body 102. For instance, in one such embodiment, a total cross-sectional area of the seal body 102 can be approximately 730 cm² and the total area of the main channel(s) 110 plus the secondary channel(s) 112 can range from 80 cm² to 160 cm². The inventors have discovered that such ratios of the cross-sectional area can be useful in preventing channel blockage as a result of blood clotting or other channel impediment.

FIG. 2 illustrates a top plan view, schematic representation of the chest seal 100 of FIG. 1 with one or more fluid sensors 200 included at the seal body 102. The one or more fluid sensors 200 included at the chest seal 100 can be configured to sense a fluid type, and the one or more fluid sensors 200 can be included at the seal body 102 such that the one or more fluid sensors 200 are configured to sense a fluid type within the main channel(s) 110 and/or the secondary channel(s) 112. For example, the fluid sensor 200 can be configured to detect carbon dioxide in a fluid stream, for instance, the fluid sensor 200 can be configured to sense a concentration of carbon dioxide in a fluid stream emanating from a chest wound and passed to the fluid sensor 200 via at least one main channel 110 and in some instance also via a secondary channel 112.

Thus, inclusion of one or more fluid sensors 200 at any of the chest seal embodiments disclosed herein can provide an integrated fluid sensor 200 for diagnostic testing of the fluid passing through the chest seal body 102 (e.g., for diagnostic testing of the fluid passing from the patient's pleural space and within the main and secondary channels 110, 112 defined at the chest seal body 102). Some embodiments can further include a user interface mechanism (e.g., a display) associated with the chest seal and configured to display data sensed by the fluid sensor(s) 200.

As shown for the example at FIG. 2, one fluid sensor 200a can be located at the seal body 102 at, or adjacent to, secondary channel 112a and another fluid sensor 200b can be located at the seal body 102 at, or adjacent to, main channel 110a. Indeed, the example of FIG. 2 shows fluid sensors 200 at, or adjacent to, each main channel 110 and secondary channel 112, though in other embodiments within the scope of this disclosure any number of fluid sensors 200 from zero, to one, to two or more can be included at the seal body 102. In some applications, it can be useful to position one or more fluid sensors 200 at or near the perimeter 116 of the seal body 102. As one such example, as shown for the exemplary embodiment at FIG. 2, one fluid sensor 200a can be located at the seal body 102 at, or adjacent to, first secondary channel outlet 119a at perimeter 116, another fluid sensor 200b can be located at the seal body 102 at, or adjacent to, first main channel outlet 115a at perimeter 116, and yet another fluid sensor 200c can be located at the seal body 102 at, or adjacent to, second secondary channel outlet 119b at perimeter 116. Fluid sensor(s) 200 positioned at, or near, the outlet of a main or secondary fluid channel at the perimeter 116 of the seal body 102 can be useful in reducing instances of blood clotting within the seal body 102 (e.g., within a main or secondary fluid channel) impeding fluid sensing functionality of the fluid sensor(s) 200.

FIG. 3 illustrates another embodiment of a chest seal 300. The chest seal 300 can be similar to, or the same as, the chest seal 100 disclosed elsewhere herein except as otherwise noted here. As such, like reference characters refer to like features disclosed elsewhere herein.

The chest seal 300 includes main channels 110a, 110b, 110c each having main channel inlet 114 at hub port 104 and respective main channel outlets 115a, 115b, 115c at perimeter 116. Chest seal 300 also includes secondary channels 112a, 112b, 112c, 112d, 112c, and 112f. Secondary channels 112a, 112b have respective secondary channel inlets 118a, 118b at, and in direct fluid communication with, main channel 110a and respective secondary channel outlets 119a, 119b at perimeter 116. Secondary channels 112c, 112d have respective secondary channel inlets 118c, 118d at, and in direct fluid communication with, main channel 110b and respective secondary channel outlets 119c, 119d at perimeter 116. And secondary channels 112e, 112f have respective secondary channel inlets 118c, 118f at, and in direct fluid communication with, main channel 110c and respective secondary channel outlets 119e, 119f at perimeter 116. The chest seal 300 is shown as including branched configurations of pairs of secondary channels 112 at a common main channel 110.

The size of the main and secondary channels 110, 112 can vary depending on the application of the chest seal 300. For some examples, the cross-sectional area of the secondary channel inlets 118 can be the same as or different than one another. For example, the secondary channel inlet 118c can define cross-sectional area 318c and the secondary channel inlet 118d can define cross-sectional area 318d that is the same as or different than cross-sectional area 318c. Also, the secondary channel inlets 118c, 118d can define respective same or different cross-sectional area 318c, 318d that each differ from a cross-sectional area 3 of main channel 110 (e.g., the secondary channel inlets 118c, 118d can define respective same or different cross-sectional area 318c, 318d that each differ from at least cross-sectional area 3 of main channel 110b). For instance, secondary channel 112c can define cross-sectional area 318c along at least a portion of a length of secondary channel 112c between the secondary channel inlet 118c and secondary channel outlet 119c, and main channel 110b can define cross-sectional area 3 along at least a portion of a length of main channel 110b between main channel inlet 114 and main channel outlet 115b where the cross-sectional area 3 is equal to or different than the cross-sectional area 318c (e.g., cross-sectional area 3 is greater than cross-sectional area 318c; cross-sectional area 3 is less than cross-sectional area 318c; cross-sectional area 3 is equal to cross-sectional area 318c).

FIG. 4 illustrates another embodiment of a chest seal 400. The chest seal 400 can be similar to, or the same as, the chest seal 100 disclosed elsewhere herein except as otherwise noted here. As such, like reference characters refer to like features disclosed elsewhere herein.

The chest seal 400 includes main channels 110a, 110b, 110c each having main channel inlet 114 at hub port 104 and respective main channel outlets 115a, 115b, 115c at perimeter 116. Chest seal 400 also includes secondary channels 112a, 112b, and 112c. Secondary channel 112a has secondary channel inlet 118a at, and in direct fluid communication with, main channel 110a and secondary channel outlets 119a at perimeter 116. Secondary channels 112b has secondary channel inlet 118b at, and in direct fluid communication with, main channel 110b and secondary channel outlets 119b at perimeter 116. And secondary channel 112c has secondary channel inlet 118c at, and in direct fluid communication with, main channel 110c and secondary channel outlets 119c at perimeter 116. Thus, the chest seal 400 is shown as including a single secondary channel 112 in direct fluid communication with each main channel 110 and, thus, the chest seal 400 does not include branched configurations of pairs of secondary channels 112 at a common main channel 110.

As shown for the exemplary embodiment at FIG. 4, for a given secondary channel 112, the cross-sectional area of the secondary channel inlet 118 and the cross-sectional area of the secondary channel outlet 119 can be the same or different. In particular, FIG. 4 shows one example where, for a given secondary channel 112, the cross-sectional area of the secondary channel inlet 118 and the cross-sectional area of the secondary channel outlet 119 are different. For example, as shown at FIG. 4, for secondary channel 112a, first secondary channel inlet 118a can have cross-sectional area 1 and first secondary channel outlet 119a can have a different cross-sectional area 3. FIG. 4 shows such an example where, for secondary channel 112a, the cross-sectional area 1 of the first secondary channel inlet 118a is less than the cross-sectional area 3 of the first secondary channel outlet 119a. In another example, the inverse configuration can be included where, for secondary channel 112a, the cross-sectional area 1 of the first secondary channel inlet 118a is greater than the cross-sectional area 3 of the first secondary channel outlet 119a.

Chest seal embodiments and features disclosed herein can help improve fluid flow through the seal body to allow for appropriate drainage of fluid from an anatomical site (e.g., a chest wound) to one or more outlets at a perimeter of the seal body. For example, the disclosed configurations of the main and secondary channels can help to prevent or reduce instances of blood clot blockage within one or more channels at the seal body to thereby prevent or reduce instances of fluid channel blockage at the chest seal which could otherwise act to impede intended fluid drainage through the chest seal and/or impede fluid sensing at the chest seal.

More specifically, the disclosed configurations of the main and secondary channels at the chest seal embodiments herein can thus help to drain fluid (e.g., blood and/or air, such as carbon dioxide) through the chest seal which, in turn, can help to improve the chest seal's ability to vent fluid (e.g., air) effectively and thereby treat pneumothorax while also providing an improved main and secondary fluid channel configuration that can improve fluid sensing capability at the chest seal. For instance, the disclosed configurations of the one or more main channels and one or more secondary channels can be configured at the seal body to help separate different fluids—for instance to help separate blood and carbon dioxide—expired from an open chest wound by, for example, helping to ensure that at least one or more main channels and/or one or more secondary channels do not fill with one type of fluid (e.g., blood in applications of the chest seal embodiment to hemopneumothorax or pleural effusion) and, thereby, help to reduce the chance of complete blockage of another type of fluid flow (e.g., air, such as CO₂) at the chest seal due to clotting of the first fluid type (e.g., blood) within the chest seal which would prohibit the chest seal from effectively venting out the other fluid type (e.g., air). Moreover, chest seal embodiments disclosed herein can be configured to help separate different fluids (e.g., blood and air) within the chest seal body via the disclosed configurations of the main and secondary channels without using mechanically actuated component(s) (e.g., without using a moving component, such as without using a mechanically actuated valve), which, in turn, can help to reduce cost and ease manufacturing.

Various examples have been described. These and other examples are within the scope of the following claims.

What is claimed is:

1. A chest seal comprising:
a seal body that includes a first seal body side and a second seal body side that is opposite the first seal body side;
a hub port defined the seal body and extending from the first seal body side toward the second seal body side;
at least one main channel defined at the seal body and in fluid communication with the hub port, the at least one main channel including a main channel inlet at the hub port and a main channel outlet at a perimeter of the seal body; and
a first secondary channel defined at the seal body, the first secondary channel including a first secondary channel inlet at the at least one main channel and a first secondary channel outlet at the perimeter of the seal body, the first secondary channel outlet being spaced apart about the perimeter of the seal body from the main channel outlet.

2. The chest seal of claim 1,
wherein the first secondary channel inlet is in direct fluid communication with the at least one main channel, and
wherein the first secondary channel inlet is in indirect fluid communication with the hub port via the direct fluid communication with the at least one main channel.

3. The chest seal of claim 1, further comprising:
a second secondary channel defined at the seal body, the second secondary channel including a second secondary channel inlet at the at least one main channel and a second secondary channel outlet at the perimeter of the seal body.

4. The chest seal of claim 3, wherein the first secondary channel outlet is spaced apart about the perimeter of the seal body from the second secondary channel outlet.

5. The chest seal of claim 4,
wherein the first secondary channel extends along the seal body in a first radial direction from the first secondary channel inlet to the first secondary channel outlet at the perimeter of the seal body, and
wherein the second secondary channel extends along the seal body in a second radial direction from the second secondary channel inlet to the second secondary channel outlet at the perimeter of the seal body, the second radial direction being different than the first radial direction.

6. The chest seal of claim 4,
wherein the first secondary channel inlet is in direct fluid communication with the at least one main channel, and wherein the first secondary channel inlet is in indirect fluid communication with the hub port via the direct fluid communication with the at least one main channel, and
wherein the second secondary channel inlet is in direct fluid communication with the at least one main channel, and wherein the second secondary channel inlet is in indirect fluid communication with the hub port via the direct fluid communication with the at least one main channel.

7. The chest seal of claim 6,
wherein the first secondary channel inlet intersects the at least one main channel at a first location along the main channel spaced apart from both the main channel inlet and the main channel outlet, and
wherein the second secondary channel inlet intersects the at least one main channel at a second location along the main channel spaced apart from both the main channel inlet and the main channel outlet, the second location being spaced radially about the seal body from the first location.

8. The chest seal of claim 6,
wherein the first secondary channel inlet intersects the at least one main channel at a first location along the main channel spaced apart from both the main channel inlet and the main channel outlet, and
wherein the second secondary channel inlet intersects the at least one main channel at the first location along the main channel spaced apart from both the main channel inlet and the main channel outlet.

9. The chest seal of claim 8,
wherein the first secondary channel extends along the seal body in a first radial direction from the first secondary channel inlet at the first location to the first secondary channel outlet at the perimeter of the seal body, and
wherein the second secondary channel extends along the seal body in a second radial direction from the second secondary channel inlet at the first location to the second secondary channel outlet at the perimeter of the seal body, the second radial direction being different than the first radial direction.

10. The chest seal of claim 6,
wherein the first secondary channel inlet and the first secondary channel outlet define a same cross-sectional area, and
wherein the second secondary channel inlet and the second secondary channel outlet define a same cross-sectional area.

11. The chest seal of claim 6,
wherein the first secondary channel inlet and the first secondary channel outlet define different cross-sectional areas, and
wherein the second secondary channel inlet and the second secondary channel outlet define different cross-sectional areas.

12. The chest seal of claim 6,
wherein the first secondary channel inlet defines a first cross-sectional area and the second secondary channel inlet defines a second cross-sectional area that is different than the first cross-sectional area.

13. The chest seal of claim 6,
wherein the first secondary channel defines a first cross-sectional area along at least a portion of a first secondary channel length between the first secondary channel inlet and the first secondary channel outlet, and
wherein the at least one main channel defines a second cross-sectional area along at least a portion of a main channel length between the main channel inlet and the main channel outlet, the first cross-sectional area being different than the second cross-sectional area.

14. The chest seal of claim 5,
wherein the at least one main channel is a first main channel, the main channel inlet is a first main channel inlet, and the main channel outlet is a first main channel outlet, and
wherein the chest seal further comprises: a second main channel, the second main channel defined at the seal body, the second main channel including a second main channel inlet at the hub port and a second main channel outlet at the perimeter of the seal body, the second main channel outlet being spaced apart about the perimeter of the seal body from the first main channel outlet.

15. The chest seal of claim 14,
wherein the first main channel is in direct fluid communication with the hub port at the first main channel inlet, and wherein the first main channel extends a first radial direction along the seal body from the first main channel inlet to the first main channel outlet, and
wherein the second main channel is in direct fluid communication with the hub port at the second main channel inlet, and wherein the second main channel extends a second radial direction along the seal body from the second main channel inlet to the second main channel outlet, the first radial direction being different than the second radial direction.

16. The chest seal of claim 15,
wherein the first secondary channel inlet is in direct fluid communication with the first main channel and in indirect fluid communication with the hub port via the direct fluid communication with the first main channel, and
wherein the second secondary channel inlet is in direct fluid communication with the second main channel and in indirect fluid communication with the hub port via the direct fluid communication with the second main channel.

17. The chest seal of claim 1, further comprising:
at least one fluid sensor at the seal body.

18. The chest seal of claim 17, wherein the at least one fluid sensor is positioned at at least one of the main channel outlet at the perimeter of the seal body and the first secondary channel outlet at the perimeter of the seal body.

19. The chest seal of claim 18,
wherein the at least one fluid sensor comprises a first fluid sensor and a second fluid sensor,
wherein the first fluid sensor is positioned at the main channel outlet at the perimeter of the seal body, and
wherein the second fluid sensor is positioned at the first secondary channel outlet at the perimeter of the seal body.

20. The chest seal of claim 1,
wherein the first seal body side comprises an adhesive,
wherein the hub port is located at a center of the seal body, and
wherein the hub port extends longitudinally from the center of the seal body at the first seal body side toward the second seal body side.
